(19) United States
(12) Patent Application Publication
(10) Pub. No.: US 2026/0098242 A1
(43) Pub. Date: Apr. 9, 2026

(54) Method for Producing Fungal Spores, Liquid Medium Used Therein and Substance Produced Thereby

(71) Applicant: Biovirid B.V., VeenendaAl (NL)

(72) Inventors: Van De Zilver Eric, Wageningen (NL); Laurens Van Leeuwen, Amsterdam (NL)

(21) Appl. No.: 19/114,954

(22) PCT Filed: Sep. 26, 2023
(86) PCT No.: PCT/EP2023/076557

§ 371 Date: Mar. 25, 2025

(30) Foreign Application Priority Data
Sep. 26, 2022 (EP) 22197711.9

(51) Int. Cl. C12N 3/00; C12N 1/14

(52) U.S. Cl. CPC C12N 3/00; C12N 1/14

(57) ABSTRACT

A method for producing fungal spores, wherein the fungal spores are grown in a first liquid medium and then in a second liquid medium, the first liquid medium having a lower carbon content than the second liquid medium.

The present invention relates to a method for producing fungal spores, a liquid medium used therein and a substance produced thereby.

More particularly, the present invention concerns a method for producing fungal spores, of the type wherein the fungal spores are grown in a liquid medium.

In agriculture and gardening, a beneficial organism is any organism that benefits the plant growing process. Plant growth regulators and biostimulants are becoming a primary research field to improve plant growth and development. Types of beneficial organisms include, for example, insects, arachnids, other animals, plants, bacteria, fungi, viruses, and nematodes. The benefits they provide may include pest control, pollination, and/or maintenance of soil health. One important category of beneficial organism is beneficial fungal microorganism. They can typically be used as microorganism biostimulants for improving height, dry weight, leaf area and/or fruit yield of crops, or as microorganism biofungicide for controlling the activity of plant pathogenic fungi and bacteria.

Typical fungi species families that are suitable for use as beneficial microorganism include, for example, Ascomycota, Basidiomycota, and Oomycota. A major challenge to the successful commercialization of active fungal microorganisms has been the difficulties in the development of cost-efficient, high-yield, commercial-scale processes that enable the production of large quantities of fungal spores with excellent properties, including, for example, stability, manufacturability, activity, and/or infectivity.

Changes in fungal membrane composition significantly affect colonization and infection. The fungal membrane is a critical component of the fungal cell that plays a crucial role in various biological processes, including adhesion, nutrient uptake, communication, and protection. Alterations in the fungal membrane impact the ability of the fungus to interact with its environment and establish successful colonization or infection.

Distinct fungal colours are attributed to the pigments it contains, such as melanin and other secondary metabolites. These pigments are involved in various biological processes, including protection against environmental stressors like UV radiation.

The present invention seeks to provide improved methods and liquid media for the cultivation and/or production of beneficial fungal spores, especially Trichoderma spores and Beauveria bassiana spores.

Thereto, the present invention according to a first independent aspect relates to a method for producing fungal spores. The fungal spores are grown in a first liquid medium and then in a second liquid medium, wherein the first liquid medium has a lower carbon content than the second liquid medium.

Processes for producing fungal spores usually fall into two major categories: solid state fermentation and liquid or submerged fermentation. In liquid fermentation processes, fungal cultures are inoculated or grown into the relevant liquid medium for the growth of spores. Different fungal species may have different growth requirements like nutrients, pH, osmotic conditions and temperature. Typically, a liquid medium comprises a carbon source in order to meet the basic requirements, for example the synthesis of carbohydrates, for the production of fungal spores. Conventionally, it was believed that liquid media suitable for producing fungal spores need to have a high carbon content. In other words, the liquid media needed to be carbon-rich. The inventors of the present invention found that, surprisingly, using a low carbon content liquid medium before using a higher carbon content liquid medium produces spores with improved bio-activities and/or improved yields.

Using the first liquid medium preferably causes priming of the fungal spores, more particularly of the fungal culture or fungal mycelium, which may be inoculated in the first liquid medium to sporulate. Priming refers to a process wherein an organism, in this case the fungal culture or mycelium, is pre-exposed for a limited amount of time to stress, which leads to an increased adaptive response to subsequent exposures. The inventors have found that by using the first liquid medium, such priming can be induced. Priming can be observed by changes in the membrane composition of the fungal spores being produced, such as the α-glucans, β-glucans, chitin and chitosan, each representing the stage the fungus is in. In particular, α-1,3-glucans are polysaccharides composed of glucose molecules linked together by α-1,3-glycosidic bonds. They are found in the cell walls of most fungi and play a role in their virulence and infectivity. The inventors have surprisingly found that priming may increase α-1,3-glucans contents in the fungal cell walls and/or cause increased pigmentation.

Preferably, the fungal spores are carbon starved in the first liquid medium. Carbon starvation in fungi occurs when they lack sufficient carbon sources, hindering growth and potentially reducing productivity. Fungi undergo physiological changes to adapt to carbon starvation, such as altering gene expression and metabolic pathways. The inventors have surprisingly found that carbon starvation can be used as a way to prime the fungal culture or fungal mycelium. In the case of the present invention, the carbon starvation in the first liquid medium leads to an increased sporulation in the second liquid medium compared to conventional production methods using a single carbon-rich liquid medium. The inventors have surprisingly found that carbon starvation plays an important role in triggering extensive sporulation. By lowering easily accessible sugars such as glucose and inoculating the fungus in a carbon limited environment, the fungus, more particularly the fungus hyphae, can be primed.

Preferably, the first liquid medium is a liquid culture medium and/or the second liquid medium is a liquid culture medium. A liquid culture medium is a medium in which the fungal spores are cultured.

The first liquid medium may contain no carbon content. In a preferred alternative, the first liquid medium does contain carbon, but with a carbon content that is preferably less than about 1.5% w/v, less than about 1.4% w/v, less than about 1.3% w/v, less than about 1.2% w/v, less than about 1.1% w/v, more preferably less than about 1.0% w/v, less than about 0.9% w/v or less than about 0.8% w/v, based on the total volume of the first liquid medium. It may be that the first liquid medium contains carbon, but with a carbon content of less than about 0.7% w/v, less than about 0.6% w/v, less than about 0.5% w/v, less than about 0.4% w/v or less than about 0.3% w/v, based on the total volume of the first liquid medium. In a most preferred embodiment, the carbon content of the first liquid medium is about 0.8% w/v based on the total volume of the first liquid medium.

Optionally, the first liquid medium contains a nitrogen source, which may or may not be in addition to a carbon source. For example, the nitrogen source may be in the form of ammonia (NH₄), nitrate (NO₃) urea ((NH₂)₂CO), pepton, yeast extract and/or soy flour. In the case of a nitrogen source, the first liquid medium may preferably have a carbon to nitrogen (C/N) ratio of no more than about 30, no more than about 25, no more than about 20, no more than about 18, no more than about 16, no more than about 14, no more than about 12 or no more than about 10. More preferably, the first liquid medium has a C/N ratio of no more than about 9, most preferably no more than about 8. More particularly, the first liquid medium may have a C/N ratio of about 30, preferably about 22, more preferably about 15 and most preferably about 8. Alternatively, the first liquid medium contains no carbon content, in which case the C/N ratio of the first liquid medium becomes zero.

In a particularly preferred embodiment, the first liquid medium contains a carbon source, preferably a complex carbon source, such as a pepton, yeast extract and/or soy flour. A “complex” carbon source refers to a carbon source that contains a mixture of different carbon-containing compounds, as opposed to simple carbon sources like glucose or sucrose, which have a single type of carbon compound. Soy flour and other complex carbon sources make affordable and balanced liquid medium for the cultivation of fungal spores, more particularly for priming of fungal mycelium and triggering sporulation.

The carbon source, preferably a soy flour and/or other complex carbon source, may be present in the first liquid medium in an amount between about 0.5% and about 5% w/v, preferably between about 0.5% and about 3% w/v, more preferably between about 1.0% and about 2% w/v and most preferably about 1.5% w/v, based on the total volume of the first liquid medium. In a particular embodiment, the carbon source comprises or is soy flour. Preferably, this soy flour is full fat and/or toasted. This means that the fat and/or the oil are kept in the soy flour. Such liquid medium containing soy flour proved to be a particularly beneficial carbon poor liquid medium in terms of obtaining improved properties and/or improved yields. More preferably, the soy flour is full fat and toasted.

Preferably, the first liquid medium contains no mono or di-saccharides.

Optionally, the first liquid medium and/or the second liquid medium comprises a salt solution and/or an antifoam agent.

Salt solutions are beneficial for survival for the spores. The salt solution may be selected from the group consisting of a sulphate, such as K₂SO₄ or MgSO₄ solution, a potassium solution, phosphate solution, such as KH₂PO₄ solution, a chloride solution, such as a KCl or a MgCl₂ solution, and combinations thereof. Salt solutions may also act as a pH buffer for the medium. One exemplary salt solution suitable for use in the method of the present invention is a solution of K₂SO₄ and MgSO₄, or KH₂PO₄ and MgSO₄.

Generally, any antifoam that is not harmful for the cultivated fungus would be suitable. Typical antifoam agents may be selected from the group consisting of alcohols, for example cetostearyl alcohols, insoluble oils, for example castor oil, stearates, polydimethylsiloxanes and other silicones derivatives, ether, glycols, and combinations thereof. One exemplary antifoam agent suitable for use in the method of the present invention is an antifoam agent based on alkoxylated fatty acid esters, preferably on a vegetable base.

The first liquid medium and/or second liquid medium may contain a solvent. The solvent may be water, preferably distilled water.

According to the first aspect of the present invention, the second liquid medium has a higher carbon content than the first liquid medium. The second liquid medium may have a carbon content of more than about 0.6% w/v, more than about 0.8% w/v, more than about 1.0% w/v, more than about 1.2% w/v, more than about 1.4% w/v, more than about 1.6% w/v, more than about 1.8% w/v, more than about 2.0% w/v, more than about 2.2% w/v or more than about 2.4% w/v, based on the total volume of the second liquid medium. Preferably, the second liquid medium has a carbon content of at least about 2.5% w/v, at least about 2.6% w/v, at least about 2.7% w/v, at least about 2.8% w/v, at least about 2.9% w/v, or at least about 3.0% w/v, based on the total volume of the second liquid medium.

Preferably, the second liquid medium has a carbon to nitrogen (C/N) ratio of at least about 20, at least about 22, at least about 24, or at least about 26. More preferably, the second liquid medium has a C/N ratio of at least about 28, for example at least about 30. In some embodiments, the second liquid medium contains no nitrogen, by which the C/N ratio becomes infinite.

The difference in carbon content, expressed in “% w/v”, between the first liquid medium and the second liquid medium may be at least about 1.0, at least about 1.1, at least about 1.2, at least about 1.3, at least about 1.4, at least about 1.5, at least about 1.6, at least about 1.7, at least about 1.8 or at least about 1.9. Preferably, the difference in carbon content, expressed in “% w/v”, between the first liquid medium and the second liquid medium is at least about 2.0, or at least about 2.1, for example at least about 2.2.

In a particular embodiment, the second liquid medium may be obtained by adding a carbon-containing source to the first liquid medium. By doing so, the composition of the medium may be adjusted without having to separate the spores from the first liquid medium.

Alternatively, the second liquid medium may be prepared separately, with the fungus being transferred from the first liquid medium to the second liquid medium at the end of the growth period in the first liquid medium.

Optionally, the second liquid medium may contain a sugar, for example a monosaccharide or a disaccharide. Optionally, the sugar is selected from the group consisting of glucose, fructose, sucrose, lactose and maltose. Preferably, the sugar is glucose. Preferably, the second liquid medium only contains mono saccharides.

The fungal spores may be grown in the first liquid medium for about 24 to about 72 hours, preferably about 30 to about 60 hours, more preferably about 40 to about 50 hours and most preferably about 48 hours. The fungal spores may be grown in the second liquid medium for about 24 to about 72 hours, preferably about 30 to about 60 hours, more preferably about 40 to about 50 hours and most preferably about 48 hours. It allows the fungal spores time to adjust, settle and grow in the respective medium. More particularly, the first medium allows the fungus to settle, grow and starve, which is referred to as the “carbon starvation”, whereas the second medium is rich in carbon allowing the fungus to complete the development of the fungal spores. The growth period in the first liquid medium may be the same or substantially the same as, or different than the growth period in the second liquid medium.

Preferably, the first liquid medium and/or the second liquid medium is aerated, preferably continuously aerated. Aeration helps with the even distribution of the content in the reactor, the interaction between the spores and the medium, thereby increasing the spore production. The aeration rate may be at from 0.1 to 10 vvm, preferably from 0.1 to 5.0 vvm, more preferably from 0.1 to 2.0 vvm, for example at about 0.15 vvm.

The inventors have found that the following possible parameters values, either separately or combined, are optimal for the growth of the fungal spores:
the temperature of the first liquid medium may be between 25° C. and 30° C.;
the temperature of the second liquid medium may be between 25° C. and 30° C.;
the pH of the first liquid medium may be between 3.0 and 6.0, between 4.0 and 6.0 or preferably about 5.0; and/or
the pH of the second liquid medium may be between 3.0 and 6.0, between 4.0 and 6.0 or preferably about 5.0.

The temperature and/or the pH of the first liquid medium may be substantially the same as, or may be different from, the temperature and/or the pH of the second liquid medium.

Optionally, the fungal spores may be pre-treated in a preculture medium prior to the growth in the first liquid medium. The purpose of this pre-feeding step is to initiate the growth of the fungus. Preferably, the preculture medium comprises glucose. Optionally, the preculture medium may comprise a YPD (yeast pepton dextrose) media, or a Sabouraud media.

Preferably, the preculture medium has a carbon content of at least about 0.5% w/v, at least about 0.6% w/v, at least about 0.7% w/v, at least about 0.8% w/v, at least about 0.9% w/v, or at least about 1.0 %w/v, based on the total volume of the preculture medium. Optionally, the preculture medium has a carbon content of no more than about 1.5% w/v, no more than about 1.4% w/v, no more than about 1.3% w/v, or no more than about 1.2% w/v, based on the total volume of the preculture medium. For example, the preculture medium has a carbon content between about 0.6% w/v and about 1.3 % w/v, between about 0.7% w/v and about 1.2% w/v, or between about 0.8% w/v and about 1.1 % w/v, based on the total volume of the preculture medium.

The preculture medium may contain carbon and/or nitrogen. Preferably, the preculture medium has a carbon to nitrogen ratio of between about 20 and about 50, between about 25 and about 45, or between about 30 and about 40. More preferably, the preculture medium has a carbon to nitrogen ratio of about 35.

The fungal spores of the present invention may be of any fungi species, preferably fungi species that may be used as a beneficial microorganism. For example, the fungal spores may be spores of Ampelomyces, Beauveria bassiana, Chaetomium, Epicoccum, Gliocladium, Muscodor, Myrothecium, Penicillium, Trichoderma, Ulocladium, Phlebiopsis, Pythium, or a combination thereof.

Preferably, the fungal spores are Trichoderma spores. Trichoderma is particularly useful in significantly suppressing the growth of plant pathogenic microorganisms and regulate the rate of plant growth. Alternatively, the fungal spores are Beauveria bassiana spores. Beauveria bassiana can be used as a biological insecticide to control a number of pests such as termites and whiteflies.

The fungal spores produced by the method of the present invention may be in the form of a powder, such as formulated in the form of a powder. Alternatively, they can be granules or seed coats. The fungal spores produced by the method of the present invention may be used as a biocontrol agent and/or a biostimulating agent. For example, they may be used for improving root development, flowering, fruit nutritional quality, abiotic stress tolerance, and/or overall growth, of plants/crops. The fungal spores produced by the method of the invention may be used as seed treatment. For example, they may be used as dry coating, seed dressing, film coat, encrustment, and/or pellet. Alternatively or additionally, the fungal spores produced by the method of the invention may be used as root treatment. For example, it may be used to produce a solution for soaking of the roots, or for spraying onto the roots, of a plantation/crop.

Preferably, the method of the present invention is a submerged liquid fermentation process.

Preferably, the method is at least partially carried out in a bioreactor. Optionally, the bioreactor may be made of metal or polyethylene.

In one embodiment of the present invention, the fungal spores relate to Trichoderma and the method is carried out so that the yield obtained is more than about 1.0E+10 spores per gram carbon, preferably more than about 1.5E+10 spores per gram carbon, more than about 2.0E+10 spores per gram carbon, more than about 2.5E+10 spores per gram carbon, more than about 3.0E+10 spores per gram carbon, more preferably at least about 3.5E+10 spores per gram carbon, or at least about 4.0E+10 spores per gram carbon, based on the grams of carbon per litre of the second liquid medium. The yield can also be expressed in a different way, namely in terms of grams of spores (dry spore mass) per gram substrate, which is defined as the organic material present in the liquid media (in total) used to grow the fungal spores. Then, the method can be carried out so that the yield obtained is more than about 0.30 grams of spores per gram substrate, preferably more than about 0.35 grams of spores per gram substrate, more than about 0.40 grams of spores per gram substrate, more than about 0.45 grams of spores per gram substrate or most preferably about 0.50 grams of spores per gram substrate.

This yield obtained in relation to Trichoderma fungal spores is inventive per se, irrespective of the use of two liquid media with one having a lower carbon content than the other. Therefore, the present invention according to an independent second aspect relates to a method for producing fungal spores, wherein the fungal spores relate to Trichoderma and are grown in a carbon-containing liquid medium so that a yield of more than about 1.0E+10 spores per gram carbon, more than about 1.5E+10 spores per gram carbon, more than about 2.0E+10 spores per gram carbon, more than about 2.5E+10 spores per gram carbon, more than about 3.0E+10 spores per gram carbon, and more preferably at least about 3.5E+10 spores per gram carbon, or at least about 4.0E+10 spores per gram carbon, is obtained, based on the grams of carbon per litre of the liquid medium; or, expressed differently, the yield obtained in dry spore mass is more than about 0.30 grams of spores per gram substrate, preferably more than about 0.35 grams of spores per gram substrate, more than about 0.40 grams of spores per gram substrate, more than about 0.45 grams of spores per gram substrate or most preferably about 0.50 grams of spores per gram substrate. In some preferred embodiments, the yield obtained for Trichoderma is more than about 6.0E+10 spores per gram carbon, or even more than about 8.0E+10 spores per gram carbon, based on the grams of carbon per litre of the liquid medium. Optionally, the Trichoderma fungal spores are grown in more than one carbon-containing liquid media and the yield as specified above is calculated based on the grams of carbon per litre of the liquid medium having the highest carbon content.

In another embodiment of the present invention, the fungal spores relate to Beauveria bassiana and the method is carried out so that the yield obtained is more than about 1.0E+10 spores per gram carbon, preferably more than about 1.5E+10 spores per gram carbon, more than about 2.0E+10 spores per gram carbon, more than about 2.5E+10 spores per gram carbon, more than about 3.0E+10 spores per gram carbon, more than about 3.5E+10 spores per gram carbon, and more preferably at least about 4.0E+10 spores per gram carbon, at least about 4.5E+10 spores per gram carbon, at least about 5.0E+10 spores per gram carbon, at least about 5.5E+10 spores per gram carbon, or at least about 6.0E+10 spores per gram carbon, based on the grams of carbon per litre of the second liquid medium. The yield can also be expressed in a different way, namely in terms of grams of spores (dry spore mass) per gram substrate, which is defined as the organic material present in the liquid media (in total) used to grow the fungal spores. Then, the method can be carried out so that the yield obtained is more than about 0.30 grams of spores per gram substrate, preferably more than about 0.35 grams of spores per gram substrate, more than about 0.40 grams of spores per gram substrate, more than about 0.45 grams of spores per gram substrate or most preferably about 0.50 grams of spores per gram substrate.

This yield that is obtained in relation to Beauveria bassiana fungal spores is inventive per se, irrespective of the use of two liquid media with one having a lower carbon content than the other. Therefore, the present invention according to an independent third aspect relates to a method for producing fungal spores, wherein the fungal spores relate to Beauveria bassiana and are grown in a carbon-containing liquid medium so that a yield of more than about 1.0E+10 spores per gram carbon, more than about 1.5E+10 spores per gram carbon, more than about 2.0E+10 spores per gram carbon, more than about 2.5E+10 spores per gram carbon, more than about 3.0E+10 spores per gram carbon, more than about 3.5E+10 spores per gram carbon, and more preferably at least about 4.0E+10 spores per gram carbon, at least about 4.5E+10 spores per gram carbon, at least about 5.0E+10 spores per gram carbon, at least about 5.5E+10 spores per gram carbon, at least about 6.0E+10 spores per gram carbon, is obtained, based on the grams of carbon per litre of the liquid medium; or, expressed differently, the yield obtained in dry spore mass is more than about 0.30 grams of spores per gram substrate, preferably more than about 0.35 grams of spores per gram substrate, more than about 0.40 grams of spores per gram substrate, more than about 0.45 grams of spores per gram substrate or most preferably about 0.50 grams of spores per gram substrate. In some embodiments, the Beauveria bassiana fungal spores are grown in more than one carbon-containing liquid media and the yield as specified above is calculated based on the grams of carbon per litre of the liquid medium having the highest carbon content.

The method according to the second and/or third aspect of the present invention may comprise any feature described herein in relation to the method of the first aspect of the present invention, in any possible combination.

The first liquid medium, which may be used to obtain priming and/or carbon starvation described herein, is inventive per se. Therefore the present invention according to an independent fourth aspect relates to a liquid medium for producing fungal spores. The liquid medium may have one or more of the features of the first liquid medium as described herein. This liquid medium stimulates the growth of fungal spores and/or helps to improve properties and bioactivities of the spores cultivated. The present invention according to a fifth independent aspect also relates to the use of the liquid medium of the fourth aspect to produce fungal spores.

The present invention according to a sixth independent aspect relates to a substance containing the fungal spores produced by the method according to the first, second and/or third aspect of the present invention, or produced using the liquid medium according to the fourth aspect of the present invention.

The inventors have found that the fungal spores produced by the methods according to the present invention, or the substance containing these spores, may have beneficial properties, such as in terms of the mass attenuation coefficient and/or the amount of 1, 3 α-glucans. These properties are inventive per se and, therefore, the invention also relates to such properties as defined below.

The present invention according to a seventh independent aspect relates to a substance containing fungal spores. The substance has a first mass attenuation coefficient at 380-480 nanometre (nm) with an adsorption of at least 1.5 or 2 litre per gram per centimetre (L·g⁻¹cm^{31 1}), more preferably above 2 L·g⁻¹cm⁻¹, and a second mass attenuation coefficient at 630-730 nm with an absorption of at least 1.0 or 1.2 L·g⁻¹cm⁻¹, more preferably above 1.5 L·g⁻¹cm⁻¹. Preferably, the first mass attenuation coefficient is at 410-450 nm, most preferably at about 430 nm. Preferably, the second mass attenuation coefficient is at 660-700 nm, most preferably at about 680 nm.

Mass attenuation coefficient is a measurement of how strongly a substance absorbs or scatters light at a given wavelength, per unit mass. The unique mass attenuation coefficient profile of the substance of the present invention gives the substance a distinctive color and improves the protection against sunlight. The substance of the present invention therefore has better stability and longer shelf life, when compared to those obtained from conventional processes.

Preferably, the fungal spores relate to Trichoderma, for example Trichoderma harzianum spores. Trichoderma harzianum spores with the unique mass attenuation coefficient profile show distinctive dark green color, and have improved effects as biostimulants, in particular on plant and/or crop root growth.

The substance according to the seventh aspect of the present invention with the unique mass attenuation coefficient as discussed above may be prepared or obtained by the method according to the first, second and/or third aspect of the invention as described herein, or produced by using the liquid medium according to the fourth aspect of the present invention.

The present invention according to an independent eight aspect relates to a substance containing fungal spores. The fungal spores have cell walls containing 1, 3 α-glucans in an amount so that the fungal spores are infectious. Infectivity is an important feature of fungi microorganisms. Higher infectivity is usually associated with improved affinity with the roots, improved effectiveness, and increased beneficial activities. 1, 3 α-glucans in the fungal cell walls contribute to successful plants infection. In one embodiment of the present invention, the substance comprises fungal spores relating to Trichoderma and the 1, 3 α-glucan content in the cell wall of the fungal spores is at least 0.2% w/w, more preferably at least 0.4% w/w or at least 0.8% w/w of the dry weight of the spores. Most preferably, the 1, 3 α-glucan content is about 0.8% w/w. In another embodiment of the present invention, the substance comprises fungal spores relating to Beauveria bassiana and the 1, 3 α-glucan content in the cell wall of the fungal spores is at least about 0.2% w/w, more preferably at least 0.4% w/w or at least 0.8% w/w of the dry weight of the spores. Most preferably, the 1, 3 a-glucan content is about 0.8% w/w. In a further independent aspect of the present invention, it relates to a substance comprising fungal spores relating to Trichoderma and having a 1, 3 α-glucan content in the cell wall of the fungal spores of at least about 0.8% w/w of the dry weight of the spores. In a still further independent aspect of the present invention, it relates to a substance comprising fungal spores relating to Beauveria bassiana and having a 1, 3 a-glucan content in the cell wall of the fungal spores of at least about 0.8% w/w of the dry weight of the spores.

The substance according to the eight aspect, or the further independent aspects, of the present invention with the unique 1, 3 α-glucans content as discussed above may be prepared or obtained by the method according to the first, second and/or third aspect of the invention as described herein, or produced using the liquid medium according to the fourth aspect of the present invention.

The present invention according to an independent ninth aspect relates to a biological control agent or biostimulating agent containing the substance according to the sixth, seventh, eight and/or further aspect of the present invention as described herein. The present invention according to another independent aspect also relates to the use of the biological control agent or the biostimulating agent according to the ninth aspect of the present invention in plant disease control or as plant growth promotor.

Any feature that has been described above in relation to any one aspect or embodiment of the invention is also disclosed hereby in relation to all other aspects and embodiments. Likewise, all combinations of two or more of the individual features or elements described above may be present in any aspect or embodiment. For brevity, all possible features and combinations have not been recited in relation to all aspects and embodiments, but they are expressly contemplated and hereby disclosed.

The term “about” in relation to a numerical value x means x plus or minus 10%.

The unit “% w/v” refers to the weight in grams per 100 ml of liquid.

The unit “vvm” is commonly used for aeration rate in fungi culture, and refers to the volume of air aerated into per volume of medium per minute.

The unit “% w/w” refers to weight percent.

To better illustrate the features of the invention, certain preferred embodiments are described below, as examples that are not to be understood restrictively.

### Example 1: Cultivation and Characterisation of Trichoderma Spores

A biologically pure culture of Trichoderma was used as the inoculation starter culture. It was pre-treated in a preculture medium comprising 0.5% w/v soymeal and 2.0% w/v glucose, based on the total volume of the preculture medium. The carbon content of the preculture medium was 1.04% w/v, and the carbon to nitrogen ratio in the preculture medium was 34.3. After the pre-treatment, the spores were introduced into a 3 Litre (L) bioreactor filled with a first liquid culture medium. The first liquid culture medium was a sterile medium with a pH of 5.0 and comprises the following components in distilled water (% w/v based on total volume of the medium):
Soy flour: 1.5%
KH₂PO₄·2 H₂O: 0.3%
MgSO₄·7 H₂O: 0.1%

The carbon content of the first liquid medium was 0.73% w/v. The carbon to nitrogen ratio was 8.0.

The bioreactor was isolated from the environment except for the air supply through tiny holes in the reactor and an overpressure release, which went through a 0.22 micrometre (μm) filter. All parts coming into contact with the medium were sterilised in an autoclave except for the acid and base solutions and the inoculation starter culture. The bioreactor comprises an impellor above the inlet aerating at a rate of 0.45 litres per minute (0.15 vvm) dispersing and mixing the air bubbles, creating an oxygenated environment in the entire bioreactor.

The temperature in the bioreactor was regulated with cooling water and an electronic heating element, and was maintained at 30° C. After 48 hours, the mycelium was carbon starved and an additional carbon source solution of 300 millilitre (mL) was introduced into the 3 L bioreactor, thereby changing the first liquid culture medium to the second liquid culture medium. The additional carbon source solution comprises about 66% w/v glucose monohydrate in distilled water.

The carbon content of the second liquid culture medium was 3.1% w/v. The carbon to nitrogen ratio of the second liquid medium was 34.3, and the pH of the second liquid culture medium was 5.0.

After another 48 hours and thus a total of 96 hours after the start of inoculation, the content in the bioreactor was sieved to remove mycelium and then centrifuged. The pellet obtained from centrifugation was sprayed on a fluidized bed of a carrier sugar powder to form agglomerates of the spores and the carrier. The agglomerates were then sieved to form the final product, which is a dark green powder.

The yield was determined by dividing the spore content (as determined by serial diluting the suspension on YPD plates) by the amount of carbon contained per litre of the second liquid medium. Additionally, spore cultures where sieved, purified and transferred to pre-weighted containers and dried in an oven over night at 80° C. Dry cell weight was measured and related to total amount of the substrate, i.e. the organic material containing glucose and soy flour used.

The yield achieved by the method as described in Example 1 was about 0.5 grams of dry spore mass per gram substrate equivalent to 4.17E+10, often 6.25E+10, and sometimes 8.34E+10, spores per gram carbon.

A comparative example was also tested. Trichoderma spores were obtained using the same method as above, except that no first liquid culture medium was used. The yield was only about 0.05 grams of dry spore mass per gram substrate equivalent to 1.0E+10 spores per gram carbon, based on the amount of carbon contained per litre of the liquid medium used.

The absorption spectrums of the innovative product and the comparative example were obtained. The spores produced by the method according to the invention had a much darker green color than the spores produced using a conventional liquid fermentation method.

Spores are purified by centrifugation, three times. Afterwards, they are incubated in 1.7 M Sodium hydroxide for 20 minutes at 0° C. Afterwards the suspension is neutralised and treated with amyloglucosidase and invertase. This reduces 1, 3 α-glucan to free D-glucose, which can then be measured using glucose oxidase and peroxidase, yielding quinoneimine which can be spectrophotometrically be measured at 510 nm and is linearly related to the 1, 3α-glucan content on the cell wall.

The 1,3 α-glucan content achieved by the method as described in Example 1 was about 0.8% w/w of the dry weight of the spores.

### Example 2—Cultivation and Characterization of Beauveria Bassiana Spores

The same production method was used to grow the Beauveria bassiana spores as described above in relation to the production of the Trichoderma spores, except that the growth rate for Beauveria bassiana is slightly reduced, making the optimal growth time in the first media 72 hours.

The yield was more than 0.5 grams of dry spore mass per gram substrate equivalent to 6.0E+10 spores per gram carbon, as determined by the method described in Example 1.

The 1,3 α-glucan content achieved by the method as described in Example 2 was about 0.8% w/w of the dry weight of the spores.

A comparative example of was also tested. Beauveria bassiana spores were obtained using the same method as above, except that no first liquid medium was used. The yield was only 0.05 grams of dry spore mass per gram substrate equivalent to 1.0E+10 spores per gram carbon.

Furthermore, the present invention also relates to the following preferred embodiments without being limited thereto:
1.—A method for producing fungal spores, wherein the fungal spores are grown in a first liquid medium and then in a second liquid medium, the first liquid medium having a lower carbon content than the second liquid medium.
2.—The method of paragraph 1, wherein the first liquid medium is used to prime the fungal spores, more particularly a fungal culture or mycelium that is preferably inoculated in the first liquid medium to sporulate.
3.—The method of paragraph 1 or 2, wherein the fungal spores are carbon starved in the first liquid medium.
4.—The method of any of the preceding paragraphs, wherein the first liquid medium is a liquid culture medium and/or the second liquid medium is a liquid culture medium.
5.—The method of any of the preceding paragraphs, wherein the first liquid medium contains no carbon; or wherein the first liquid medium contains carbon and preferably has a carbon content of less than about 1.5% w/v, less than about 1.4% w/v, less than about 1.3% w/v, less than about 1.2% w/v, less than about 1.1% w/v, less than about 1.0% w/v, less than about 0.9% w/v, less than about 0.8% w/v, less than about 0.7% w/v, less than about 0.6% w/v, less than about 0.5% w/v, less than about 0.4% w/v or less than about 0.3% w/v, based on the total volume of the first liquid medium.
6.—The method of any of the preceding paragraphs, wherein the first liquid medium has a carbon to nitrogen ratio of no more than about 30, no more than about 25, no more than about 20, no more than about 18, no more than about 16, no more than about 14, no more than about 12, no more than about 10, no more than about 9 or no more than about 8.
7.—The method of any of the preceding paragraphs, wherein the first liquid medium contains a carbon source, preferably a complex carbon source, such as pepton, yeast extract and/or soy flour.
8.—The method of paragraph 7, wherein the carbon source, preferably soy flour and/or other complex carbon source, is present in the first liquid medium in an amount of between about 0.5% and about 5% w/v, preferably between about 0.5% and about 3% w/v, more preferably between about 1.0% and about 2% w/v and most preferably about 1.5 % w/v, based on the total volume of the first liquid medium.
9.—The method of paragraph 7 or 8, wherein the carbon source is soy flour, which preferably is full fat and/or toasted.
10.—The method of any of the preceding paragraphs, wherein the first liquid medium contains no mono or di-saccharides.
11.—The method of any of the preceding paragraphs, wherein the second liquid medium contains a carbon content of more than about 0.6% w/v, more than about 0.8% w/v, more than about 1.0% w/v, more than about 1.2% w/v, more than about 1.4% w/v, more than about 1.6% w/v, more than about 1.8% w/v, more than about 2.0% w/v, more than about 2.2% w/v or at least about 2.4% w/v, based on the total volume of the second liquid medium.
12.—The method of any of the preceding paragraphs, wherein the second liquid medium has a carbon to nitrogen (C/N) ratio of at least about 20, at least about 22, at least about 24, at least about 26, at least about 28 or at least about 30.
13.—The method of any of the preceding paragraphs, wherein the difference in carbon content, expressed in “% w/v”, between the first liquid medium and the second liquid medium is at least about 1.0, at least about 1.1, at least about 1.2, at least about 1.3, at least about 1.4, at least about 1.5, at least about 1.6, at least about 1.7, at least about 1.8, at least about 1.9, at least about 2.0, at least about 2.1 or at least about 2.2.
14.—The method of any of the preceding paragraphs, wherein the second liquid medium is obtained by adding a carbon-containing source to the first liquid medium.
15.—The method of any of the preceding paragraphs, wherein the second liquid medium comprises a sugar, such as glucose.
16.—The method of any of the preceding paragraphs, wherein the second liquid medium only contains mono saccharides.
17.—The method of any of the preceding paragraphs, wherein the fungal spores are grown in the first liquid medium for 24 to 72 hours, preferably for 30 to 60 hours, more preferably for 40 to 50 hours and most preferably for about 48 hours and/or the fungal spores are grown in the second liquid medium for 24 to 72 hours, preferably for 30 to 60 hours, more preferably for 40 to 50 hours and most preferably for about 48 hours.
18.—The method of any of the preceding paragraphs, wherein the first and/or second liquid medium is aerated, preferably continuously, for example at a rate of about 0.1 to 2.0 vvm, preferably at about 0.15 vvm.
19.—The method of any of the preceding paragraphs, wherein the fungal spores are grown in the first liquid medium and/or the second liquid medium at a temperature of between about 25° C. and 30° C.
20.—The method of any of the preceding paragraphs, wherein the fungal spores are grown in the first liquid medium and/or the second liquid medium at a pH of between about 3.0 and 6.0, between 4.0 and 6.0, or preferably about 5.0 pH.
21.—The method of any of the preceding paragraphs, wherein the fungal spores are pre-treated in a preculture medium prior to the growth in the first liquid medium, the preculture medium preferably having one or more of the following characteristics:
the preculture medium has a carbon content between about 0.6% w/v and about 1.3 % w/v, between about 0.7% w/v and about 1.2% w/v or between about 0.8% w/v and about 1.1 % w/v, based on the total volume of the preculture medium; and/or
the preculture medium has a carbon to nitrogen ratio of between about 20 and about 50, between about 25 and about 45, or between about 30 and about 40.
22.—The method of paragraph 21, wherein the preculture medium contains glucose.
23.—The method of any of the preceding paragraphs, wherein the fungal spores relate to Trichoderma or Beauveria, such as Beauveria bassiana.
24.—The method of any of the preceding paragraphs, wherein the fungal spores are produced as powder, granules or seed coats.
25.—The method of any of the preceding paragraphs, wherein the fungal spores serve as biocontrol agent or biostimulating agent.
26.—The method of any of the preceding paragraphs, wherein the method is a submerged liquid fermentation process.
27.—The method of any of the preceding paragraphs, wherein the method is at least partially carried out in a bioreactor.
28.—The method of any of the preceding paragraphs, wherein the fungal spores relate to Trichoderma; and the method is carried out so that the yield obtained is more than about 1.0E+10 spores per gram carbon, more than about 1.5E+10 spores per gram carbon, more than about 2.0E+10 spores per gram carbon, more than about 2.5E+10 spores per gram carbon, more than about 3.0E+10 spores per gram carbon, more preferably at least about 3.5E+10 spores per gram carbon, or more than about 4.0E+10 spores per gram carbon, based on the grams of carbon per litre of the second liquid medium; or, expressed in a different manner, the yield obtained in dry spore mass is more than about 0.30 grams of spores per gram substrate, more than about 0.35 grams of spores per gram substrate, more than about 0.40 grams of spores per gram substrate, more than about 0.45 grams of spores per gram substrate, or most preferably about 0.50 grams of spores per gram substrate.
29.—The method of any of paragraphs 1 to 27, wherein the fungal spores relate to Beauveria bassiana; and the method is carried out so that the yield obtained is more than about 1.0E+10 spores per gram carbon, preferably more than about 1.5E+10 spores per gram carbon, more than about 2.0E+10 spores per gram carbon, more than about 2.5E+10 spores per gram carbon, more than about 3.0E+10 spores per gram carbon, more than about 3.5E+10 spores per gram carbon, and more preferably more than about 4.0E+10 spores per gram carbon, at least about 4.5E+10 spores per gram carbon, at least about 5.0E+10 spores per gram carbon, at least about 5.5E+10 spores per gram carbon, or more than about 6.0E+10 spores per gram carbon, based on the grams of carbon per litre of the second liquid medium; or, expressed differently, the yield obtained in dry spore mass is more than about 0.30 grams of spores per gram substrate, preferably more than about 0.35 grams of spores per gram substrate, more than about 0.40 grams of spores per gram substrate, more than about 0.45 grams of spores per gram substrate or most preferably about 0.50 grams of spores per gram substrate.
30.—A method for producing fungal spores, wherein the fungal spores relate to Trichoderma and are grown in a carbon-containing liquid medium so that a yield of more than about 1.0E+10 per gram carbon, more than about 1.5E+10 spores per gram carbon, more than about 2.0E+10 spores per gram carbon, more than about 2.5E+10 spores per gram carbon, more than about 3.0E+10 spores per gram carbon, more preferably at least about 3.5E+10 spores per gram carbon, or more than about 4.0E+10 spores per gram carbon, is obtained, based on the grams of carbon per litre of the liquid medium used; or, expressed differently, wherein the yield obtained in dry spore mass is more than about 0.30 grams of spores per gram substrate, more than about 0.35 grams of spores per gram substrate, more than about 0.40 grams of spores per gram substrate, more than about 0.45 grams of spores per gram substrate, or most preferably about 0.50 grams of spores per gram substrate.
31.—A method for producing fungal spores, wherein the fungal spores relate to Beauveria bassiana and are grown in a carbon-containing liquid medium so that a yield of more than about 1.0E+10 spores per gram carbon, more than about 1.5E+10 spores per gram carbon, more than about 2.0E+10 spores per gram carbon, more than about 2.5E+10 spores per gram carbon, more than about 3.0E+10 spores per gram carbon, more than about 3.5E+10 spores per gram carbon, and more preferably more than about 4.0E+10 spores per gram carbon, at least about 4.5E+10 spores per gram carbon, at least about 5.0E+10 spores per gram carbon, at least about 5.5E+10 spores per gram carbon, or more than about 6.0E+10 spores per gram carbon is obtained, based on the grams of carbon per litre of the liquid medium used; or, expressed differently, wherein the yield obtained in dry spore mass is more than about 0.30 grams of spores per gram substrate, preferably more than about 0.35 grams of spores per gram substrate, more than about 0.40 grams of spores per gram substrate, more than about 0.45 grams of spores per gram substrate or most preferably about 0.50 grams of spores per gram substrate.
32.—The method according to paragraph 30 or 31, the method showing one or more of the features as defined in any of paragraphs 1 to 29.
33.—A liquid medium for producing fungal spores, having one or more of the features of the first liquid medium as defined in any of the paragraphs 1-32.
34.—Use of the liquid medium of paragraph 33 to produce fungal spores.
35.—A substance containing fungal spores produced by the method of any of the paragraphs 1-32, or produced using the liquid medium of paragraph 33.
36.—A substance containing fungal spores and having a first mass attenuation coefficient at about 380-480 nm of at least about 2 Lg⁻¹cm⁻¹ and a second mass attenuation coefficient at about 630-730 nm with an absorption of at least about 1.2 or 1.5 Lg⁻¹cm⁻¹.
37.—The substance of paragraph 36, wherein the first mass attenuation coefficient is at about 410-450 nm, preferably at about 430 nm.
38.—The substance of paragraph 36 or 37, wherein the second mass attenuation coefficient is at about 660-700 nm, preferably at about 680 nm.
39.—The substance of any of paragraphs 36 to 38, wherein the fungal spores relate to Trichoderma, for example Trichoderma harzianum spores.
40.—A substance containing fungal spores having cell walls containing 1, 3 a-glucans in an amount so that the fungal spores are infectious.
41.—A substance containing fungal spores, wherein the fungal spores relate to Trichoderma and the 1, 3 α-glucan content in the cell wall is at least about 0.8% w/w of the dry weight of the spores.
42.—A substance containing fungal spores, wherein the fungal spores relate to Beauveria bassiana and the 1, 3 α-glucan content is at least about 0.8% w/w of the dry weight of the spores.
43.—A biological control agent or biostimulating agent containing the substance according to any of the paragraphs 35 to 42.
44.—Use of the biological control agent or biostimulating agent of paragraph 43 in plant disease control or as plant growth promotor.

The present invention is in no way limited to the embodiments described above, but such methods, liquid media and substances may be realised of different variants without going beyond the scope of the present invention.

1. A method for producing fungal spores, comprising growing the fungal spores in a first liquid medium and then in a second liquid medium, the first liquid medium having a lower carbon content than the second liquid medium.

2. The method of claim 1, wherein the first liquid medium is used to prime the fungal spores.

3. The method of claim 1, wherein the fungal spores are carbon starved in the first liquid medium.

4. The method of claim 1, wherein the first liquid medium has a carbon content of less than about 1.5% w/v, less than about 1.4% w/v, less than about 1.3% w/v, less than about 1.2% w/v, less than about 1.1% w/v, less than about 1.0% w/v, less than about 0.9% w/v or less than about 0.8% w/v, based on a total volume of the first liquid medium.

5. The method of claim 1 , wherein the first liquid medium has a carbon to nitrogen ratio of no more than about 30, no more than about 25, no more than about 20, no more than about 18, no more than about 16, no more than about 14, no more than about 12, no more than about 10, no more than about 9 or no more than about 8.

6. The method of claim 1, wherein the first liquid medium contains a carbon source.

7. The method of claim 6, wherein the carbon source is present in the first liquid medium in an amount of between about 0.5% and about 5% w/v, preferably between about 0.5% and about 3% w/v, more preferably between about 1.0% and about 2% w/v and most preferably about 1.5% w/v.

8. The method of claim 7, wherein the carbon source is soy flour.

9. The method of claim 1, wherein the second liquid medium contains a carbon content of more than about 0.6% w/v, more than about 0.8% w/v, more than about 1.0% w/v, more than about 1.2% w/v, more than about 1.4% w/v, more than about 1.6% w/v, more than about 1.8% w/v, more than about 2.0% w/v, more than about 2.2% w/v or at least about 2.4% w/v, based on a total volume of the second liquid medium.

10. The method of claim 1, wherein the second liquid medium has a carbon to nitrogen ratio of at least about 20, at least about 22, at least about 24, at least about 26, at least about 28 or at least about 30.

11. The method of any claim 1, wherein the fungal spores are pre-treated in a preculture medium.

12. The method of claim 1, wherein the fungal spores relate to Trichoderma; wherein the method is carried out so that a yield obtained is more than about 1.0E+10 spores per gram carbon, more than about 2.0E+10 spores per gram carbon, or more than about 4.0E+10 spores per gram carbon, based on the grams of carbon per litre of the second liquid medium; or, wherein a yield obtained in dry spore mass is more than about 0.30 grams of spores per gram substrate, more than about 0.35 grams of spores per gram substrate, more than about 0.40 grams of spores per gram substrate, or about 0.50 grams of spores per gram substrate; or wherein the 1, 3 α-glucan content in the cell wall is at least about 0.8% w/w of the dry weight of the spores.

13. The method of claim 1, wherein the fungal spores relate to Beauveria bassiana; wherein the method is carried out so that a yield obtained is more than about 1.0E+10 spores per gram carbon, more than about 4.0E+10 spores per gram carbon, more than about 6.0E+10 spores per gram carbon, based on the grams of carbon per litre of the second liquid medium; or, wherein a yield obtained in dry spore mass is more than about 0.30 grams of spores per gram substrate, more than about 0.35 grams of spores per gram substrate, more than about 0.40 grams of spores per gram substrate, more than about 0.45 grams of spores per gram substrate or about 0.50 grams of spores per gram substrate; or wherein the 1, 3 α-glucan content in the cell wall is at least about 0.8% w/w of the dry weight of the spores.

14. canceled

15. A substance containing fungal spores having a first mass attenuation coefficient at about 380-480 nm, at about 410-450 nm, or at about 430 nm, of at least about 2 Lg⁻¹cm⁻¹ and a second mass attenuation coefficient at about 630-730 nm, at about 660-700 nm, or at about 680 nm, with an absorption of at least about 1.2 or 1.5 Lg^{31 1}cm⁻¹.

16. The substance of claim 15, wherein the fungal spores relate to Trichoderma.

17. The substance of claim 16, wherein the fungal spores include Trichoderma harzanium spores.

18. A liquid medium for producing fungal spores, comprising one or more of a carbon source, a yeast extract, a soy flour, or another complex carbon source.

19. The liquid medium of claim 18, wherein the carbon source is pepton.

20. The liquid medium of claim 18, wherein the soy flour is full fat or toasted.

21. The method of claim 11, wherein the preculture medium comprises glucose.
